(19) United States
(12) Patent Application Publication
(10) Pub. No.: US 2025/0345574 A1
(43) Pub. Date: Nov. 13, 2025

(54) SYSTEM, APPARATUS, AND METHOD FOR AUTOMATED DRAINING OF URINE

(71) Applicant: Mohammad Amin Shakhaje, I, Yazd (IR)

(72) Inventor: Mohammad Amin Shakhaje, I, Yazd (IR)

(73) Assignee: Mohammad Amin Shakhaje, I, Yazd (IR)

(21) Appl. No.: 18/869,265

(22) PCT Filed: May 26, 2022
(86) PCT No.: PCT/IR2023/050011

§ 371 Date: Nov. 25, 2024

(51) Int. Cl. A61M 27/00; A61M 25/00; A61M 25/10

(52) U.S. Cl. CPC A61M 27/00; A61M 25/0023; A61M 25/1002; A61M 25/1011; A61M 25/10182; A61M 2025/0001; A61M 2025/105; A61M 2025/1052; A61M 2025/1061; A61M 2202/0496; A61M 2205/3334; A61M 2205/3344; A61M 2210/1085

(57) ABSTRACT

This application relates to a system, device, and method for detecting and draining urine. The catheter uses at least two teardrop-shaped retention balloons and a balloon-blocking mechanism to allow efficient and complete urine drainage away from the user after urination.

In another embodiment, urine is automatically detected by a sensor and drawn away from the user by a pump to a storage location.

### CROSS-REFERENCE TO RELATED APPLICATION

This disclosure is a continuation-in-part of PCT international application PCT/IR2023/050011, filed on May 26, 2023, entitled ‘SYSTEM, APPARATUS, AND METHOD FOR AUTOMATED DRAINING OF URINE’, which claims priority from the granted IR patent application 1 Patent Application Ser. No. 1401501400030001628, filed on May 26, 2022, which are incorporated herein by reference in its entirety.

### TECHNICAL FIELD

This disclosure relates to the field of medical devices. More specifically, it relates to a system, apparatus, and method for automated draining urine by eliminating urinary incontinence problems and the incidence of bacterial infections in catheterized patients.

### BACKGROUND

The novel features which are believed to be characteristic of the present disclosure, as to its structure, organization, use and method of operation, together with further objectives and advantages thereof, will be better understood from the following drawings in which a presently preferred embodiment of the present disclosure will now be illustrated by way of example. It is expressly understood, however, that the drawings are for illustration and description only and are not intended as a definition of the limits of the present disclosure. Embodiments of the present disclosure will now be described by way of example in association with the accompanying drawings in which:

The indwelling or intermittent catheterization of the bladder has historically been viewed as safe because of the antiseptic methods of use and manufacture that have become common practice. Catheters are utilized for several different clinical reasons such as urinary retention, urinary incontinence, and following lower urinary tract surgery. It is well known in the art that using external catheters can cause a variety of serious problems for the user. Some of the main problems include large residual volume remaining in the bladder, frequent blockage of the catheter, and the incidence of bacterial infections in catheterized patients that virtually all patients who undergo long-term catheterization become infected. Many of these complications occur frequently when the catheter is not able to drain the urine completely.

In view of these problems, there have been a number of attempts in the prior art to solve these problems.

It has been found that the current design of urine catheters can result in a large residual volume remaining in the bladder or elsewhere in the drainage system such as the drain tube that connects the catheter to the drainage bag.

Retention balloons for self-sustaining the catheter within the bladder could be one of the main reasons for the large residual volume remaining in the bladder.

Catheter in the prior art utilizes single or two balloons located on a catheter tube wherein the balloon fully surrounds the catheter tube. In the catheter with two balloons, one balloon is placed above the other balloon.

For example, a single, expandable balloon that projects past the catheter tip may slip out of the bladder and does not allow for complete draining of the bladder because there is no distal urine inlet at or below the distal side of the single balloon. Besides, locating the hole below the single balloon will not allow for continuous drainage due to the bladder wall closing around the single balloon. Moreover, placing the urine inlet above the single balloon would not allow for complete draining, because the balloon completely surrounds the proximal end of the catheter thus urine is trapped below this single balloon and a residual volume of urine would be retained in the bladder. In addition to the above-mentioned drawbacks, the prior art retention balloons have large surface areas which are prone to the formation of biofilms on their surfaces acting as breeding grounds for the proliferation of microorganisms within the bladder and increasing the risk of infection. In this invention, two retention balloons can be configurated like a spherical member connected to a stand with a curved lateral surface, such that urine flows into the urine drainage tube through the curved space between the balloon stand and the catheter body. In addition, the retention balloons have small surface areas causing less infection risk by providing fewer breeding grounds for the proliferation of microorganisms within the bladder.

It has been also found that the current design of catheters, can result in the risk of solidification of urine in the urine drainage tube, frequent blockage of the catheter, and following that the incidence of bacterial infections. These problems are typically addressed with simple procedures such as flushing or manual manipulation. However, on many occasions, these procedures fail and replacement of the catheter becomes necessary.

The remaining urine in the urine drainage tube could be the result of incomplete urine drainage, blockage of the catheter, or closing the urine drainage tube at the proximal end of the catheter, which may cause a standing column of urine that may back right up into the patient. It is also widely accepted that to control infection, fluid that has drained from a patient should not be allowed to flow back to the patient.

In this invention, certain provisions have been made to solve the above-mentioned problems, including closing, cleaning, and disinfecting the urine tube. A blocking mechanism that is configured to occlude and close the urine drainage tube, is positioned just under the distal urine inlet in the distal end of the catheter causing an empty urine tube in which the pharmaceutically active agents and/or washing medium are delivered.

Another problem with the current urinary catheters is that when the urine fluid that has drained from a patient is allowed to flow back to the patient. In order to solve this problem, some urine suction devices, which typically transport urine from a patient to a tank or reservoir using pumps are operated. In this invention, a controlling system is programmed to control complete draining by a suction pump, such that the urine drainage tube is occluded by this controlling system based on measuring the pressure of the bladder, and the medical and/or washing fluid is delivered into the tube.

The prior art catheters and methods of use do not provide a controlling device that simultaneously enables: preventing the incidence of bacterial infections, allowing complete automated drainage of urine, preventing blockage of urine tube, allowing the directional flow of medicament into the bladder and urine tube as desired, and measuring of the urine property through a catheter device which is controlled automatically.

The device and method of the current invention described below address these deficiencies and problems and further provide a combination catheter to improve urine output measurement and urine parameter analysis. The urine drainage catheter itself offers an untapped opportunity to collect and measure additional patient parameters.

### SUMMARY

The following brief summary is not intended to include all features and aspects of the present application, nor does it imply that the application must include all features and aspects discussed in this summary.

In one general aspect, the application describes a medical device with a new design in retention balloon, a specific fluid draining and delivering medical agents' systems, and a method for fluid draining and delivering medical the same.

A medical device according to one embodiment of the present invention may include: a catheter (100) comprising a distal portion (102), a central portion (104), and a proximal portion (106), one or more openings defined at the distal portion, with a plurality of lumens placed along the longitudinal axis of the catheter (100), wherein each of the plurality of lumens may have a distal end in the distal portion and a proximal end in the proximal portion of the catheter (100);

In one aspect, the plurality of the lumens comprises a urine drainage lumen (120), a restriction lumen (130), a balloon inflation lumen (140), a medicine supply lumen (150), and a pressure-sensing lumen (160). The urine drainage lumen (120) may be in fluid communication with at least two distal openings (124) at the distal portion (102) and also, with a proximal port (122) at the proximal portion (106) of the catheter (100), wherein the urine is withdrawn via the urine drainage lumen (120) through the at least two distal openings (124).

In one aspect, the balloon inflation lumen (140) may be in fluid communication with at least two retention balloons (144) at the distal portion (102) and also, with a proximal balloon port (142) at the proximal portion (106) of the catheter (100), wherein each retention balloon (144) can be mounted on an edge of one of the at least two distal openings (124) in the distal portion (102) of the catheter (100). Moreover, the at least two retention balloons (144) may have a teardrop shape with a concavely curved lateral surface on one end. Moreover, in order to inflate the retention balloons (144) while keeping the catheter in the bladder, a crescent-shaped lumen (146) may be coupled with the at least two retention balloons (144) at the distal portion (102) and also may be coupled with the balloon inflation lumen (140) at the central portion (104) of the catheter to pass the sterile liquid or air via the inflation lumen (140) to the at least two retention balloons (144).

In one aspect, the medicine supply lumen (150) may be in fluid communication with a plurality of infusion outlets (154) at the distal portion (102) of the catheter (100) and a plurality of inner slits (156) on the sidewall of the urine drainage lumen (120), wherein the medicine supply lumen (150) can be in fluid communication with the urine drainage lumen (120) via the multiple inner slits (156). Moreover, the medicine supply lumen (150) may be in the fluid communication with a proximal infusion port (152) at the proximal portion (106) of the catheter (100). Moreover, a second crescent-shaped lumen (155) having an inner surface and an outer surface may be coupled with the distal end of the medicine supply lumen (150), wherein the outer surface of the crescent-shaped lumen (155) can be coupled with the plurality of infusion outlet (154 and the inner surface of the crescent-shaped lumen (155) can be coupled with the plurality of inner slits (156).

In one aspect, the lumen (130) may be positioned next to the urine drainage lumen (120), wherein the urine drainage lumen (120) and the restriction lumen (130) may be divided with an inner wall (135). Moreover, the thickness of the inner wall (135) is thinner in the central portion (104) of the catheter than in the proximal portion (106), wherein the thinner inner wall (135) of the central portion (104) is closer to the distal portion (102) rather than the proximal portion (106). Moreover, the distal end of the lumen (130) may be closed at the thinner inner wall (135) portion.

In one aspect, the catheter (100) may further comprise a blocking balloon (132) positioned within the restriction lumen (130), wherein once the blocking balloon (132) passes the restriction lumen (130), the blocking balloon (132) enters into the urine drainage lumen (120), due to the closed distal end of the lumen (130) at the thinner inner wall (135) portion.

In one aspect, the pressure-sensing balloon (164) may be positioned at the distal portion (102) of the catheter (100) and may be in fluid communication with a pressure-sensing lumen (160). Moreover, the pressure-sensing lumen (160) may be in fluid communication with a pressure port (162) at the proximal portion (106) of the catheter (100).

In one aspect, the medical device may further include a controlling device (200) that may be coupled with the proximal portion (106) of the catheter (100) through a plurality of fluid lines (602, 603, 604, and 605). The controlling device (200) may comprise a syringe system (500) to spray medical agent and washing fluid into the urine drainage lumen (120) and/or the bladder, and a balloon inflation system (400) to inflate and deflate the pressure-sensing balloon (164) and the blocking balloon (132). Moreover, the controlling device (200) may be configured to control a balloon-blocking mechanism (137) to block the urine drainage lumen (120).

In one aspect, a method for draining fluid, according to one embodiment of the present invention may include: positioning a catheter (100) into a body of a subject, coupling a controlling device (200) in communication with the catheter (100), inflating the blocking balloon (132) via the balloon inflation system (400) to pass through the inner wall (135) of the restriction lumen (130) to occlude the urine drainage lumen (120), filling the pressure-sensing balloon (164) via a balloon inflation system (400) to measure the bladder pressure, deflating the blocking balloon (132) to open the urine drainage lumen (120) when the pressure differential between the body subject and balloon rises above its limit state, such that the fluid is allowed to flow along the urine drainage lumen 120, inflating the blocking balloon 132 to block the urine drainage lumen when the pressure in pressure-sensing balloon (164) equals the pressure in the bladder, spraying the drug or fluid into the body of a subject and the urine drainage lumen (120).

### BRIEF DESCRIPTION OF THE DRAWINGS

The novel features which are believed to be characteristic of the present disclosure, as to its structure, organization, use, and method of operation, together with further objectives and advantages thereof, will be better understood from the following drawings in which a presently preferred embodiment of the present disclosure will now be illustrated by way of example. It is expressly understood, however, that the drawings are for illustration and description only and are not intended as a definition of the limits of the present disclosure. Embodiments of the present disclosure will now be described by way of example in association with the accompanying drawings in which:

The invention will now be described by reference to the preferred embodiments.

FIG. 1A shows a general perspective view of a catheter comprising multiple separate ports in accordance with an exemplary and non-limiting implementation;

FIG. 1B shows a general perspective view of the catheter having various sections (distal, central, and proximal), consistent with one or more exemplary embodiments of the present disclosure;

FIG. 1C shows a general perspective view of the catheter comprising inflatable retention balloons, distal infusion outlet, pressure-sensing balloon, and distal openings, consistent with one or more exemplary embodiments of the present disclosure;

FIG. 2 illustrates a transverse cross-sectional top view taken along the lines F-F shown in FIG. 1C, consistent with one or more exemplary embodiments of the present disclosure;

FIG. 3A shows a perspective view of the one deflated retention balloon mounted on one distal opening at the distal portion of the catheter, consistent with one or more exemplary embodiments of the present disclosure;

FIG. 3B shows a longitudinal cross-sectional side view of deflated retention balloons, inner slit, and distal infusion outlet of the catheter taken along the lines E-E shown in FIG. 3A, consistent with one or more exemplary embodiments of the present disclosure;

FIG. 3C shows a longitudinal cross-sectional side view taken along the lines E-E shown in FIG. 5A, consistent with one or more exemplary embodiments of the present disclosure;

FIG. 4A illustrates a transverse cross-sectional view of the distal portion of the catheter is taken along the lines B-B shown in FIG. 1C, according to one or more exemplary embodiment of the present disclosure;

FIG. 4B illustrates a transverse cross-sectional view of the distal portion of the catheter showing a fluid communication between a balloon inflation lumen and retention balloons taken along the lines B-B shown in FIG. 1C, consistent with one or more exemplary embodiments of the present disclosure;

FIG. 5A illustrates a perspective view of the distal and central portion of the catheter, consistent with one or more exemplary embodiments of the present disclosure;

FIG. 5B illustrates a transverse cross-sectional view taken along the lines G-G shown in FIG. 5A, consistent with one or more exemplary embodiments of the present disclosure;

FIG. 5C illustrates a transverse cross-sectional view taken along the lines G-G shown in FIG. 5A, consistent with one or more exemplary embodiments of the present disclosure;

FIG. 6A illustrates a perspective view of the catheter with balloon blocking mechanism, consistent with one or more exemplary embodiments of the present disclosure;

FIG. 6B illustrates a longitudinal cross-sectional view of an open condition of balloon blocking mechanism, consistent with one or more exemplary embodiments of the present disclosure;

FIG. 6C illustrates a longitudinal cross-sectional view of a close condition of balloon blocking mechanism, consistent with one or more exemplary embodiments of the present disclosure;

FIG. 7A shows a cross-sectional side view of a pressure-sensing balloon coupled to the pressure-sensing lumen, consistent with one or more exemplary embodiments;

FIG. 7B shows a cross-sectional side view of an expanded pressure-sensing balloon coupled to the pressure-sensing lumen, consistent with one or more exemplary embodiments;

FIG. 8 shows a perspective view of a controlling device coupled to the proximal portion of the catheter, consistent with one or more exemplary embodiments;

FIG. 9 shows a perspective exploded view of a syringe system, consistent with one or more exemplary embodiments;

FIG. 10 shows a perspective view of a controlling device, consistent with one or more exemplary embodiments;

FIG. 11 shows a perspective exploded view of a balloon inflation system, consistent with one or more exemplary embodiments;

### DETAILED DESCRIPTION

The novel features which are believed to be characteristic of the present disclosure, as to its structure, organization, use, and method of operation, together with further objectives and advantages thereof, will be better understood from the following discussion.

Aspects of the invention are illustrated by way of example and not by way of limitation in the figures of the accompanying drawings in which references indicate similar elements. It should be noted that references to “an” and “one” embodiment in this disclosure are not necessarily to the same embodiment, and such references mean at least one. In the following description, numerous specific details are set forth to provide a thorough description of the invention. However, it will be apparent to one skilled in the art that the invention may be practiced without these specific details. In other instances, well-known features have not been described in detail so as not to obscure the invention.

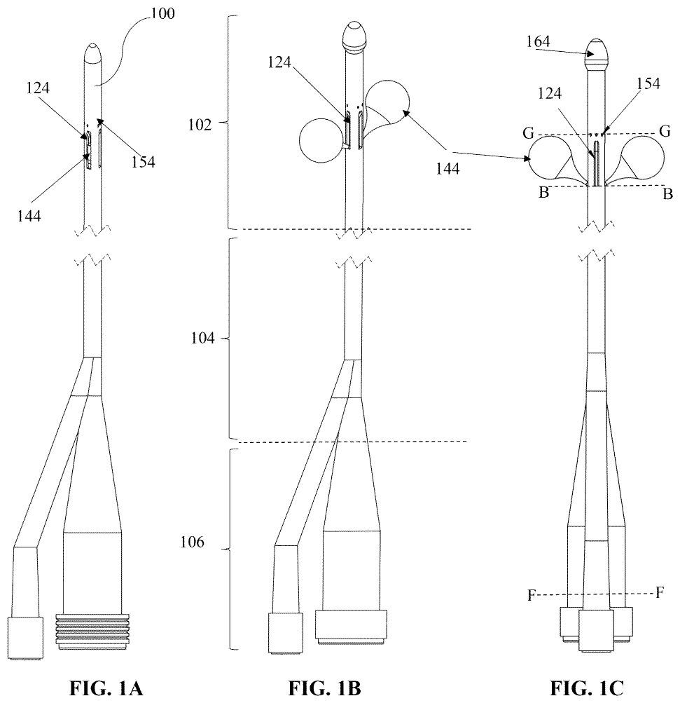

FIG. 1A illustrates an embodiment of a catheter 100 and several of its features, which may be understood to have various sections including but not limited to a distal or urinary bladder-residing portion 102 which is called hereinafter “the distal portion”, a central or urethra-residing portion 104 which is called hereinafter “central portion”, and a proximal portion 106 that remains external to the subject.

The disclosed catheter system 100 can be configured to measure the urine excretion flow, control the automatic urine drainage, and deliver pharmaceutically active agents into the bladder and/or urine drainage lumen 120, by coupling to a controlling device 200 (illustrated in FIG. 8), according to one implementation of the present disclosure.

The main elements of the disclosed catheter 100 are explained in more detail in further figures herein. The disclosed details are not intended to be limited to the implementations shown but are to be accorded the widest possible scope consistent with the principles and features disclosed herein.

Referring back to FIG. 1A, the catheter 100 can comprises one or more openings near the distal portion 102 of the catheter 100 and multiple separate ports near the proximal portion 106 of the catheter 100 in which some or all of the ports may be integrated into a single port, or as illustrated in FIG. 8, it may integrate into a urine drainage line which travels to the controlling device 200.

As a main embodiment consistent with one or more exemplary embodiments of the present disclosure, the catheter 100, may have multiple lumens which described herein passing along the longitudinal axis of the catheter as illustrated in FIG. 1A to FIG. 1C, and may be connected to the related lines in controlling device 200 which shows in FIG. 8; a urine drainage lumen 120 adapted to pass the urine into the collecting receptacle (not shown) in the controlling device 200; a restriction lumen 130 which may adapted to block the urine drainage lumen 120 via the controlling device 200; a balloon inflation lumen 140 which may adapted to convey a pressurized fluid that is used to inflate an inflatable at least two retention balloon 144 in order to keep the catheter in the bladder; a medicine supply lumen 150 which can be adapted to infuse a pharmaceutically active agents or washing fluid into the bladder through a plurality of infusion outlets 154 near the distal portion 102 of the catheter 100; and a pressure-sensing lumen 160 which can be adapted to measure the bladder pressure in order to control automatic drainage of urine.

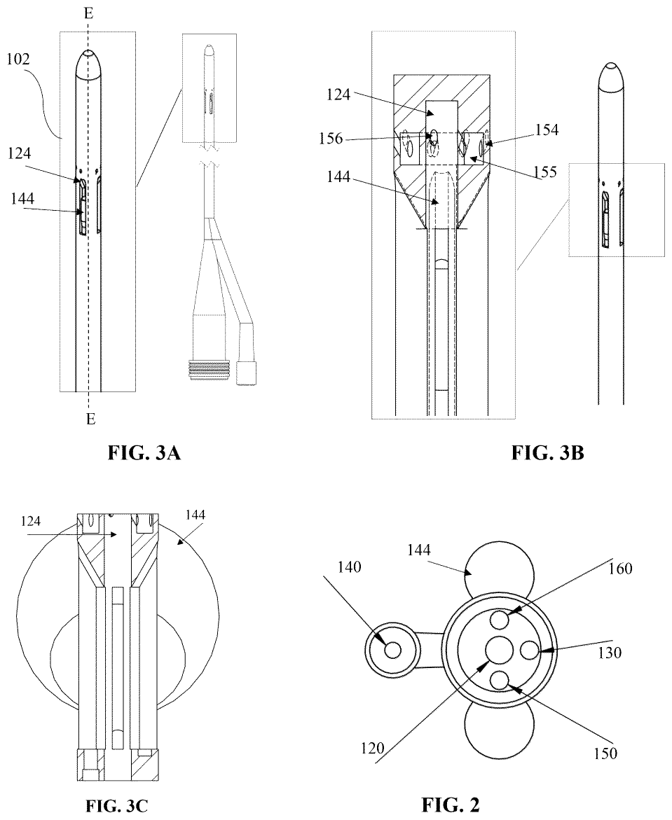

FIG. 2, clarifies in more detail what was disclosed generally in FIG. 1A to FIG. 1C and shows a transverse cross-sectional top view of the proximal portion 106 of the catheter 100 which is taken along the hypothetical lines F-F shown in FIG. 1C according to one or more exemplary embodiments of the present disclosure. FIG. 2 illustrates the embodiment of the present disclosure, in which the catheter 100 surrounding the urine drainage lumen 120, the restriction lumen 130, the medicine supply lumen 150, and the pressure-sensing lumen 160 and the at least two retention balloons 144 and a balloon port 142 positioned beside the catheter 100.

According to some exemplary embodiment of the present disclosure, as illustrated in FIG. 1A, the urine drainage lumen 120 which is placed along the longitudinal axis of the catheter may have at least two distal openings 124 that reside in the bladder-residing portion 102 of the catheter. The urine drainage lumen 120 may further have a proximal port 122 at the proximal portion 106 of the catheter as shown in FIG. 1A. The urine drainage lumen 120 in this exemplary embodiment may be in fluid-flow communication with the at least two distal openings 124 and the proximal port 122. In this embodiment, urine can be withdrawn, from the at least two distal openings 124 and drawn out the catheter 100 through the proximal port 122.

In an exemplary embodiment, in order to keep the catheter in the bladder, as shown in FIG. 1B, at least two inflatable retention balloons 144 can be mounted on one edge of the at least two distal openings 124 in the distal portion 102 of the catheter, where the at least two retention balloons 144 may be in fluid communication with the balloon inflation lumen 140 which is in fluid communication with a balloon port 142 at, or near the proximal portion 106 of the catheters.

According to different embodiments of the present disclosure mounting the at least two retention balloons 144 on the catheter 100, may result in increased diameters of the catheter. To reduce the diameter of the catheter 100, the at least two distal openings 124 need to have enough space to accommodate the at least two retention balloons 144 while the balloons are deflated.

FIG. 3A clarifies the position of one deflated retention balloon 144 inside the one distal opening 124 according to one exemplary embodiment of the present disclosure. FIGS. 3B to 3C, are a longitudinal cross-sectional view of the distal portion 102 of the catheter 100 which is taken along the hypothetical lines E-E shown in FIG. 3A according to one or more exemplary embodiment of the present disclosure. FIG. 3B and FIG. 3C receptively illustrates the position of the deflated and inflated retention balloon 144 with respect to the at least two distal openings 124.

The at least two retention balloons 144 of the catheter may be retained inside the urinary bladder and continuously contacts the mucosal lining of the inner bladder wall, which causes the remaining residual volume of urine under the at least two distal openings 124 and surrounding the at least two retention balloons 144. This residual volume of urine may cause serious problems for patients including the accumulation and proliferation of pathogenic organisms within the bladder which may render the patient susceptible to subsequent infection or harm to bladder and kidney tissue.

In order to avoid the remaining residual volume of urine under the at least two distal openings 124 and surrounding the at least two retention balloons 144, in an embodiment, as shown in FIG. 1B which some of its details are illustrated in FIG. 3A according to one or more exemplary embodiment of the present disclosure, at least two distal openings 124 may accommodate the at least two retention balloons 144 while one another at least two distal openings 124 may have no retention balloon 144 positioned on it. In this exemplary embodiment, each of the at least two retention balloons 144 may have a teardrop shape with a concavely curved lateral surface on one end. This could help flow urine into the at least two distal openings 124 through the curved space that exists between the at least two retention balloons 144 and the at least two distal openings 124. The location of the at least two retention balloons 144 besides this configuration synergically increases the urine flow rate and allow for urine drainage without the remaining residual volume of urine inside the bladder.

Referring back to FIG. 1A to FIG. 1C there is a medicine supply lumen 150 which passes inside the catheter body and along the longitudinal axis of the catheter 100 and communicates with the plurality of infusion outlets 154 at the end of the distal portion 102 of the catheter 100 and with a proximal infusion port 152 at the proximal portion 106 of the catheter 100. In this exemplary embodiment according to the present disclosure, the medicine supply lumen 150 and the plurality of infusion outlets 154 which is placed into the bladder may serve as an infusion conduit where medicinal agents, pharmaceutically active agents, or any washing fluid can be infused.

In this exemplary embodiment, the proximal infusion port 152 may be configured to allow a syringe to be coupled to the proximal infusion port 152 and deliver a medical agent through the medicine supply lumen 150 into the bladder.

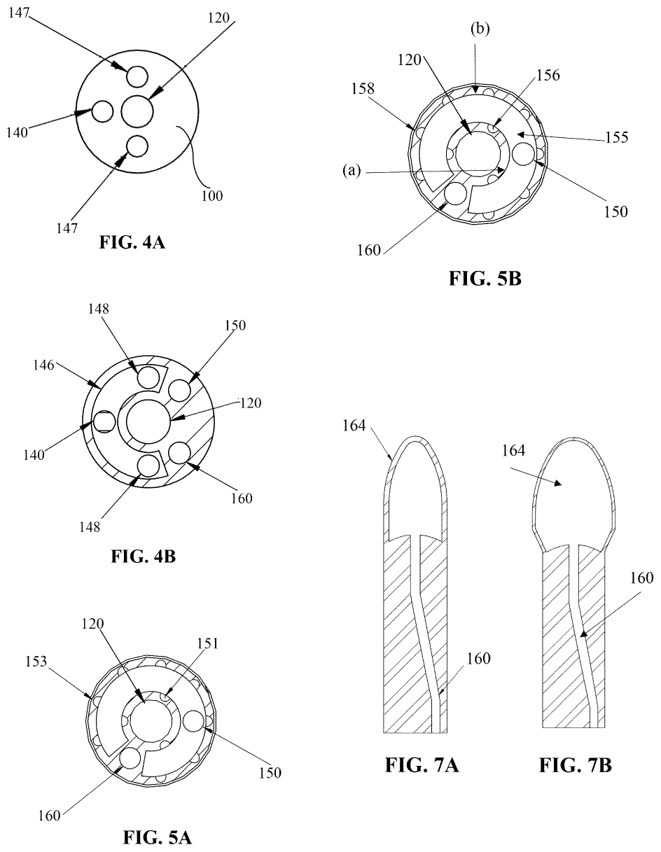

In an exemplary embodiment as illustrated in FIG. 1B, in order to inflate at least two retention balloons 144 while keeping the catheter in the bladder, the balloon inflation lumen 140 in the distal portion 102 of the catheter, may be connected to at least two smaller lumens 147. This embodiment is more clear in FIG. 4A, in which a transverse cross-sectional top view of the distal portion 102 of the catheter is taken along the lines B-B shown in FIG. 1C according to one or more exemplary embodiments of the present disclosure. As illustrated in FIG. 4A, the two smaller lumens 147 may be interconnected with the balloon inflation lumen 140, in such a way that the at least two retention balloons 144 can be inflated by the flow of sterile liquid or air via the inflation lumen 140 through smaller lumens 147. The at least two retention balloons 144 are generally attached to the smaller lumens 147, meaning that the balloons may be directly connected and fixed to the smaller lumens 147 or may be connected via various types of connecting ports and means.

FIG. 4B, illustrates another exemplary embodiment of the present disclosure. As is illustrated in this figure, there is a crescent-shaped lumen 146 at the distal portion 102 of the catheter in order to distinguish it from the other crescent-shape lumens that are explained in the following sections, it will be called the “first crescent-shape lumen” hereinafter and connected to the end segment of the balloon inflation lumen 140 which encloses a semicircular area smaller than the cross-section area of the distal portion 102 of the catheter 100. This embodiment may have two holes as fluid outlets 148 on opposing arcs at an inner boundary thereof as shown in FIG. 4B. In this exemplary embodiment, the fluid outlets 148 can be coupled with the at least two retention balloons 144. The first crescent-shaped lumen 146 allows fluid communication between the balloon port 142 and the at least two retention balloons 144.

The body fluid drainage catheters are generally faced with frequent blockage of the catheter which may result in the occurrence of bacterial infections in catheterized patients especially those who undergo long-term catheterization. In order to solve the problems of urine drainage lumen 120 clogging by fluid-borne debris and therefore elimination of bacterial infections, the urine drainage lumen 120 of the present disclosure can be unclogged and disinfected via medicinal agents or a washing fluid. The washing fluid in some implementations may include, for example, an antimicrobial drug, an antiviral drug, an anti-bacterial agent, and/or a detergent. For this purpose, as illustrated in FIG. 8, a controlling device 200 containing a syringe system 500 can be used, which configured to spray medical agent and washing fluid into the urine drainage lumen 120. According to this embodiment, the fluid flow from the syringe system 500 pass through the medicine supply lumen 150 which can be in fluid communication with the plurality of infusion outlets 154 at the distal portion 102 of the catheter 100 and a plurality of inner slits 156 on the sidewall of the urine drainage lumen 120, such that the medicine supply lumen 150 can be in fluid communication with the urine drainage lumen 120 via multiple inner slits 156. In an exemplary embodiment, as illustrated in FIG. 5A, the catheter may comprise a smaller lumen 151 which can be in fluid communication with the medicine supply lumen 150 and may be configured to couple with the plurality of inner slit 156, in which medical agent and washing fluid flow from the syringe system 500 through the smaller lumen 151 and spray into the urine drainage lumen 120.

In order to deliver medical agent and the washing fluid into the bladder and urine drainage lumen simultaneously, as illustrated in FIG. 5B, another embodiment of the present disclosure can have a crescent-shaped lumen 155 which in order to be distinguished from the other crescent-shape lumen that explained in the previous sections, is named the second crescent-shape lumen 155 hereinafter. FIG. 5B illustrates a transverse cross-sectional view taken along the lines G-G shown in FIG. 1C. The second crescent-shaped lumen 155 may have an inner surface (a) and an outer surface (b) and may be coupled with the distal end of the medicine supply lumen 150 at the distal portion 102 of the catheter, which encloses approximately a circular area equal or smaller than the cross-section area of the distal portion 102 catheters. As shown in FIG. 5B, the outer surface (b) of the second crescent-shaped lumen 155 may be coupled with the plurality of infusion outlets 154 for spraying medical agents or the washing fluid into the bladder, and the inner surface (a) of the second crescent-shaped lumen 155 may be coupled with the inner slits 156 on the sidewall of the urine drainage lumen 120 for spraying medical agents or washing fluid into urine drainage lumen 120. This embodiment may eliminate using multiple smaller lumens leading to decrease catheter diameter.

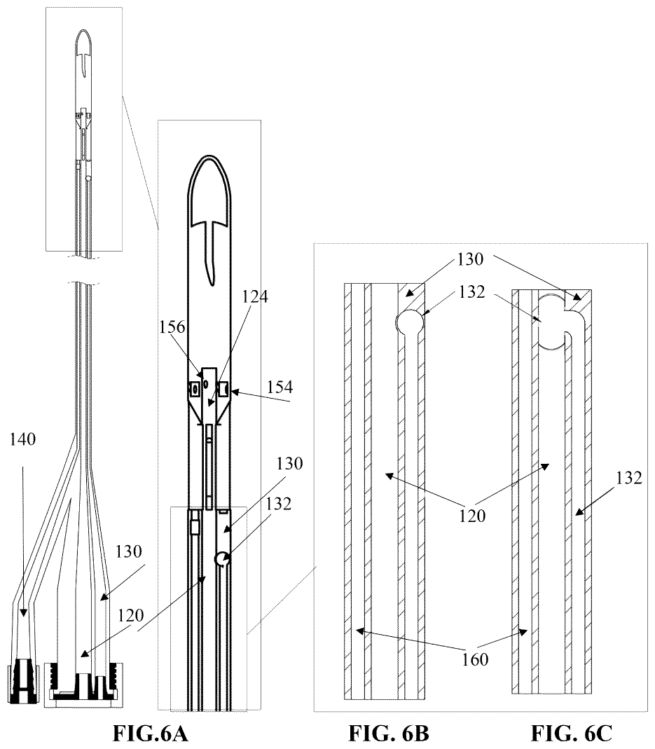

In some embodiments of the present disclosure, certain provisions have been made to solve the problem of urinary incontinence. There is an infection risk associated with the ordinary continually open urine drainage lumens, which provides a direct passage for bacteria or other microorganisms into the bladder. In order to solve this problem in the present disclosure, as illustrated in FIG. 6A to FIG. 6C, a balloon-blocking mechanism 137 can be configured to control occluding the urine drainage lumen 120 in the catheter 100. The balloon blocking mechanism 137 typically blocks the drain lumen to prevent the draining of urine through the drain lumen of the catheter when the blocking balloon 132 is engaged. The balloon-blocking mechanism 137 is positioned just under the at least two distal openings 124 in the central portion 104 of the catheter 100 and may comprise the urine drainage lumen 120, the restriction lumen 130 and the blocking balloon 132, and this balloon-blocking mechanism 137 can be connected to the controlling device 200. As shown in FIG. 6A to FIG. 6C, the urine drainage lumen 120 and restriction lumen 130 are divided with an inner wall 135, wherein the thickness of the inner wall 135 is thinner in the central portion 104 compared to the proximal portion 106 of the catheter 100. In an exemplary embodiment, as illustrated in FIG. 6A, the distal end of the lumen 130 may be closed at the thinner inner wall 135 portions.

FIG. 6B and FIG. 6C, specifically make clear the open and the close conditions happens respectively in the drainage lumen 120 as described in the previous paragraph. The blocking balloon 132 according to the same exemplary embodiment can be placed in the restriction lumen 130 and pass through that thereafter to block the urine drainage lumen 120. Once the blocking balloon 132 passes the restriction lumen 130, the blocking balloon 132 enters into the urine drainage lumen 120, due to the closed distal end of the lumen 130 at the thinner inner wall 135 portion.

Since the balloon blocking mechanism 137 is positioned just under at least two distal openings 124 in the central portion 104 of the catheter 100, a very small amount of urine remains in the urine drainage lumen 120 during the closing state of the balloon blocking mechanism 137, therefore the risk of solidification of urine in the urine drainage lumen 120 is reduced and the following incidence of bacterial infections will be controlled.

Different embodiments of the present disclosed catheter system may include the ability to measure the pressure within the bladder either via connecting one or more pressure balloons to the catheter 100 or via inserting one or more pressure balloons or pressure sensors within the respected lumens of the catheter 100. According to one exemplary embodiment of the present disclosures illustrated in FIG. 1B and FIG. 1C and with further details in FIG. 7A, in order to measure the bladder internal pressure to control automatic drainage of urine, an inflatable pressure-sensing balloon 164 may be placed at or near the distal portion of the catheter 102.

The pressure-sensing balloon 164 as illustrated in FIG. 7A may be coupled to the pressure-sensing lumen 160 which is in communication with a pressure port 162 (FIG. 1B) at the proximal portion 106 of the catheter 100. In the exemplary embodiment, as shown in FIG. 7B, the pressure-sensing balloon 164 is formed from or includes a compliant membrane, wherein the surface area of the membrane expands or contracts as a function of the expansion of the balloon.

In an exemplary embodiment, in order to measure the urine excretion flow, to control the automatic urine drainage and to deliver a pharmaceutically active agent, the distal or urinary bladder-residing portion 102 of the catheter 100 can be inserted through the urethra and into the patient's bladder and the proximal portion 106 of the catheter may be coupled to the controlling device 200 via connecting assembly 600 including a connecting ring 601 wherein the catheter lumens (120, 150 and 160) can be coupled to the fluid lines (602, 603, 604, and 605) of controlling device 200, as shown in FIG. 8. In an exemplary embodiment, the controlling device 200 could further comprises a pump syringe system 500 and a balloon inflation system 400.

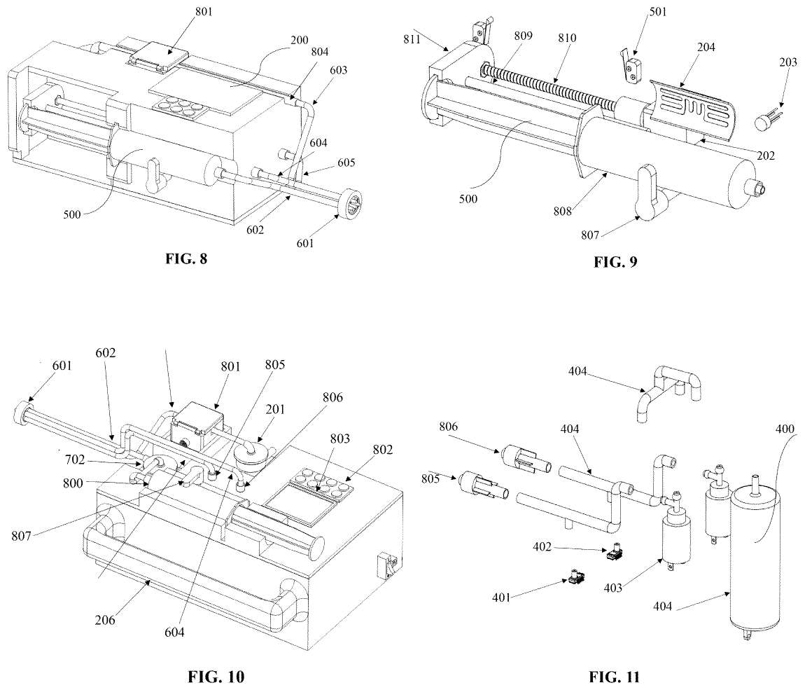

In order to deliver a pharmaceutically active agent through the medicine supply lumen 150 into the bladder, in an exemplary embodiment of the present disclosure which is illustrated in FIG. 9, the proximal infusion port 152 can be connected to the pump syringe system 500 via the line 602. In this embodiment, the pump syringe system 500 can comprise a medicine storage unit 808, a syringe holder 807, a syringe heater 204 to heat a fluid within the syringe, a step motor 202, a temperature sensor 203, a guide rod 809, a threaded rod 810, a syringe arm portion 811, and a micro-switch 501.

In an exemplary embodiment, the urine drainage lumen 120 may be connected to a line drainage 603 which is in fluid communication with a urine drainage tube 804 which is configured to convey the urine to a collecting receptacle (not shown). In this exemplary embodiment, the urine drainage tube 804 can be integrated with a flowmeter sensor 801 or it can be operated without any integration with the flowmeter sensor 801. The flowmeter sensor 801 in this exemplary embodiment is configured to measure the flow property of the urine while detecting air bubbles, water, or other clogs that may be formed into the urine drainage lumen 120. The flow property that is measured by the flowmeter sensor 801 can be at least one or more than one of the following metrics: volume, mass, or velocity of the urine passing through or proximate to the flowmeter.

The controlling device 200 (FIGS. 8-11) in some exemplary embodiments, may include a keyboard 802 to set the working mode and a screen 803 to display various metrics such as the urine emptying volume from the bladder and/or the delivered drug volume.

In another exemplary embodiment of the present disclosure, as illustrated in FIG. 11, the pressure port 162 may be connected to line 604 which is in fluid communication with a port 805 which is connected to the balloon inflation system 400. In this exemplary embodiment, the balloon inflation system 400 may include a pressure gauge sensor 401 for measuring the pressure of the bladder, a pressure gauge sensor 402 for controlling the pressure of the blocking balloon 132, an air pump 404 for inflating balloons, a plurality of communication pipes 405, and at least two electric valves 403 to control the airflow.

In an exemplary embodiment, the blocking balloon port 132 and pressure port 162 may be connected to balloon inflation system 400 via the lines 604 and 605, receptively.

Embodiments of the disclosed system and method in the present disclosure can include an automatic urine drainage system, in addition to measuring the urine excretion flow and delivering the pharmaceutically active agents by the controlling device 200. Accordingly, as illustrated in FIG. 6A to FIG. 6C when the catheter has been inserted into the bladder, the deflated blocking balloon 132 is configured to be filled with air and can be pass through the restriction lumen 130 to close the urine drainage lumen 120. When inflation fluid is applied to the deflated blocking balloon 132, the balloon is inflated to a fully expanded condition and passes through the thin inner wall 135 of the lumen 130, to block the urine drainage lumen 120. Then, the fluid can enter into the pressure-sensing lumen 160, which can result in filling the pressure-sensing balloon 164 which comprises the pressure-sensing interface to measure the bladder pressure. The amount of air volume that should enter into the pressure-sensing balloon 164 is measured by the pressure gauge sensors 402. Accordingly, a tuning system (not shown) can detect the optimum target pressure and volume to inflate the pressure-sensing balloon 164 and add or remove air or fluid volume as needed until the pressure in balloon 164 equals the pressure in the bladder.

According to explained exemplary embodiment, once the pressure in the bladder measured, a valve 806 can be opened and stayed open until the blocking balloon 132 is deflated. While the blocking balloon 132 is deflated within the restriction lumen 130, the fluid (urine, medicine medium and medical agent according to present disclosure) is allowed to flow along the urine drainage lumen 120 between the at least two distal opening 124 and the proximal port 122 and can flow out to the line drainage 603. Accordingly, as urine enters the urine drainage tube 804, the urine pump 201 may be activated to pump the urine to the collecting receptacle.

In this exemplary embodiment, the flowmeter can measure the actual volume of the urine passing through the flowmeter and when the detected volume reaches a substantially full state, the blocking balloon 132 can be inflated and block the urine drainage lumen 120. In one aspect, in order to avoid a sudden discharge of urine or/and bladder bleeding, the emptying step may be performed intermittently every 15 minutes or it is operated based on demand. So, automatic urine drainage can be controlled by measuring urine volume.

Alternatively, measuring the pressure difference between the bladder and the pressure-sensing balloon 164 is another way to control automatic urine drainage. In an exemplary embodiment, as the volume of urine increases in the bladder, the pressure on the pressure-sensing balloon 164 may also increase and when the detected pressure reaches its limit state, the urine drainage process can be permitted to flow in the urine drainage lumen 120. And as soon as the pressure in the balloon 164 equals the pressure in the bladder, which can be understood that the bladder is empty of urine and finally the process of urine drainage may be stopped. Accordingly, when the bladder is empty of urine; the drug pump may be activated to spray the drug or fluid that has reached the desired temperature by the heater 204 into the bladder and the urine drainage lumen 120, and then the bladder can be drained again.

1- A medical device, comprising:
a catheter (100) comprising a distal end portion (102), a central portion (104), and a proximal end portion (106), one or more openings defined at the distal end portion, with a plurality of lumens placed along the longitudinal axis of the catheter (100), wherein each of the plurality of lumens has a distal end in the distal end portion and a proximal end in the proximal end portion of the catheter (100); wherein the plurality of the lumens comprises:
a urine drainage lumen (120) in fluid communication with at least two distal openings (124) at the distal end portion (102), wherein urine is withdrawn via the urine drainage lumen (120) through the distal openings (124);
a balloon inflation lumen (140) in fluid communication with at least two retention balloons (144), wherein each retention balloon (144) is mounted on an edge of one of the distal openings (124) in the distal end portion (102) of the catheter (100);
a medicine supply lumen (150) in fluid communication with a plurality of infusion outlets (154) at the distal end portion (102) of the catheter (100) and a plurality of inner slits (156) on the sidewall of the urine drainage lumen (120), wherein the medicine supply lumen (150) is in fluid communication with the urine drainage lumen (120) via multiple inner slits (156).

2. The medical device of claim 1, wherein each of the retention balloons (144) has a teardrop shape with a concavely curved lateral surface on one end.

3. The medical device of claim 1, wherein the urine drainage lumen (120) is in fluid communication with a proximal drainage port (122) at the proximal end portion (106).

4. The medical device of claim 1, wherein the balloon inflation lumen (140) is in fluid communication with a proximal balloon port (142) at the proximal end portion (106) of the catheter (100).

5. The medical device of claim 1, wherein the medicine supply lumen (150) is in fluid communication with a proximal infusion port (152) at the proximal end portion (106) of the catheter (100).

6. The medical device of claim 1, wherein the catheter (100) further comprises a first crescent-shaped lumen (146) coupled with the retention balloons (144).

7. The medical device of claim 1, wherein the catheter (100) further comprises a second crescent-shaped lumen (155) having an inner surface and an outer surface coupled with the distal end of the medicine supply lumen (150).

8. The medical device of claim 7, wherein the outer surface of the second crescent-shaped lumen (155) is coupled with the plurality of infusion outlets (154).

9. The medical device of claim 6, wherein the inner surface of the second crescent-shaped lumen (155) is coupled with the plurality of inner slits (156).

10. The medical device of claim 1, wherein the catheter (100) further comprises a restriction lumen (130) positioned next to the urine drainage lumen (120).

11. The medical device of claim 10, wherein the urine drainage lumen (120) and the restriction lumen (130) are divided by an inner wall (135).

12. The medical device of claim 11, wherein the thickness of the inner wall (135) is thinner in the central portion (104) of the catheter (100) than in the proximal end portion (106).

13. The medical device of claim 10, wherein the distal end portion (102) of the lumen is closed at the thinner inner wall (135) portion.

14. The medical device of claim 12, wherein the thinner inner wall (135) of the central portion (104) is closer to the distal end portion (102) than the proximal end portion (106).

15. The medical device of claim 10, wherein the catheter (100) further comprises a blocking balloon (132) positioned within the restriction lumen (130).

16. The medical device of claim 15, wherein the blocking balloon (132) mounts on the thinner inner wall (135) of the central portion (104).

17. The medical device of claim 1, wherein the catheter (100) further comprises a pressure-sensing balloon (164) positioned at the distal end portion (102) of the catheter (100) and in fluid communication with a pressure-sensing lumen (160).

18. The medical device of claim 1, wherein the pressure-sensing lumen (160) is in fluid communication with a pressure port (162) at the proximal end portion (106) of the catheter (100).

19-26 (canceled)
